(19) United States
(12) Patent Application Publication
(10) Pub. No.: US 2014/0220643 A1
(43) Pub. Date: Aug. 7, 2014

(54) Method and Apparatus for the Production of an Arabinoxylan-Enriched Preparation and Other Co-Products

(71) Applicant: Weili Li, Manchester (GB)

(72) Inventor: Weili Li, Manchester (GB)

(73) Assignee: MANCHESTER METROPOLITAN UNIVERSITY, Manchester, Lancashire (GB)

(21) Appl. No.: 14/128,407

(22) PCT Filed: Jun. 19, 2012
(86) PCT No.: PCT/GB2012/051409

§ 371 Date: Apr. 2, 2014

(30) Foreign Application Priority Data
Jun. 21, 2011 (GB) 1110471.8

(51) Int. Cl. C12P 19/04; A23L 1/30; C12P 19/02; C08B 37/00; C13K 1/00

(52) U.S. Cl. CPC C12P 19/04; C08B 37/006; C13K 1/00; C12P 19/02; A23L 1/30
USPC 435/101; 536/123; 127/36; 435/105

(57) ABSTRACT

A method for the production of an arabinoxylan-enriched preparation from a pentosan fraction derived from a wheat flour comprising the following sequential steps: mixing the pentosan fraction with water to obtain a pentosan slurry; centrifuging the slurry to obtain liquid and solid phases; and drying the liquid phase to provide the arabinoxylan-enriched preparation. Also provided are methods for the production of: a starch- and protein-enriched intermediate from wheat flour; a glucose-enriched preparation from wheat flour; and a starch- and protein-enriched material from wheat flour. Furthermore, there is provided a use of an arabinoxylan-enriched preparation in one or more of: food, immune stimulants, prebiotics, nutraceuticals, pharmaceuticals and food supplements. There is also an apparatus for the production of an arabinoxylan-enriched preparation from a pentosan fraction of wheat flour.

### BACKGROUND

1. Technical Field

The present invention relates generally to the production of arabinoxylan-enriched preparations. More particularly, but not exclusively, the present invention concerns a method and apparatus for the production of arabinoxylan-enriched preparations and other co-products from a pentosan fraction derived from a wheat flour separation process, for example, high pressure disintegration (HPD) processing on wheat flour.

2. Description of the Related Art

High pressure disintegration (HPD) processing is one known method for the separation of starch and gluten from wheat flour by taking advantage of their differences in solubility, density and size. HPD is one of four recognised industrial processes for the separation of wheat starch and gluten: Martin; Alfa-Laval/Raisio; Hydrocyclone; and High-Pressure Disintegration (HD). The processes differ mainly in the forms of the flour-water mixtures presented to the centrifuge, hydrocyclone or screen, or in the initial separation practice of starch and gluten fractions from flour. However, the processes all purify intermediate starch and gluten-particle streams to give more than 98% of pure starch and approximately 80% of gluten.

The HPD process is based on a highly shearing a batter and using centrifugal forces for separation of different fractions. Typically, wheat flour is mixed with water at 35° C. in a continuous dough mixture to create a smooth batter that is then forced through a homogenising valve using pressures of 100 bar. This causes starch granules to be released and gluten threads to aggregate into micrometer-sized particles. The batter is diluted and then fed into a three-phase decanter centrifuge which separates the batter: first the α-starch is discharged; second the β-starch and gluten are separated into two streams for refining; and thirdly a pentosan other solubles fraction are separated from the β-starch stream. In the pentosan fraction, any remaining fine gluten particles are removed using vibrating screens.

The pentosan fraction from HPD processing is often used for ethanol production, by converting the starch to glucose and fermenting the glucose. However, the pentosan fraction is a source of arabinoxylans, which are not only thought to provide a defence against pathogens, but are also anti-oxidants and a good source of dietary fibres that function to lower cholesterol. Accordingly, arabinoxylans have become nutritionally important.

EP 2270237 A discloses a method for producing arabinoxylans by making a water-soluble arabinoxylan from wheat flour. The method comprises a sequence of complex steps and procedures as follows: (a) producing a flour-water suspension; (b) maintaining the suspension for a rest period of 3-10 minutes; (c) diluting the flour-water suspension with circulating water (101); (d) decanting the flour-water suspension by a three-phase decanter (105) in a centrifugal field; and (e) separating the resulting head water (108) in a further centrifugal field. This part of the process is the same as the HPD process. EP 2270237 A continues with the following steps: (f) recycling the heavy fraction (106) obtained in the further centrifugal field in the starch extraction cycle; (g) adding alpha-amylase to the light fraction obtained in the further centrifugal field; (h) dextrinizing the light fraction with a jet-cooker (120); (i) storing the hydrolyzate with flocculation and sedimentation in a reaction vessel for 0.3-13 hours at a pH of 1-10; (j) centrifuging the sediments; (k) grinding and drying the sediments; (l) sterilizing the cleared hydrolyzate; (m) saccharifying the sterilized, cooled and dextrinated hydrolyzate in a stirred reactor using glucoamylase with simultaneous proteolysis with a mixture of an alkaline and a neutral protease; (n) ultra-filtering the hydrolyzate by a multistage ultra-filtration, where a permeate and a retentate are obtained and the retentate volume is no more than a fifth of the initial volume; (o) evaporating the permeate until a concentration of 50-90% dry substance is reached; (p) evaporating the retentate until a concentration of 20-50% dry matter is reached; (q) recycling the two evaporation condensate in the starch process; (r) precipitating the evaporated retentate to an ethanol concentration of 60-80 mass % in a cycle that is operated in semi-batch procedure, where the ethanol is formed by a stirred reactor containing 94-99 vol. % of ethanol at 5-70[deg] C, additionally by a high speed rotor-stator-mixer, and an addition possibility of concentrated retentate in a bypass inlet of the rotor-stator mixer is under vacuum conditions; (s) filtering and washing the filter cake with 94-99 vol. % of ethanol and subsequent drying and grinding; and (t) reconditioning and recycling the ethanol used for washing.

In EP 2270237, delivery of the arabinoxylans is achieved by taking the pentosan (light) fraction and hydrolysing the starch into glucose, and subsequently separating the glucose and the arabinoxylans using multi-stage membrane filtration. Multi-stage membrane filtration of glucose and arabinoxylans is complex, costly and time-consuming and delays the delivery of the arabinoxylans.

An alternative method is utilised by US 2010-035302 A, which describes a method for producing preparations comprising soluble arabinoxylans as co-products of ethanol production through fermentation of whole-grain cereals. The method first mixes water with milled cereal material to obtain slurry, which is then liquefied into a mash in the presence of an amylase. The mash is saccharified in the presence of an amyloglucosidase, and then fermented with a micro-organism that produces mainly ethanol as a fermentation product. Once the fermented mash has been distilled to yield an ethanol fraction and a whole stillage fraction, the whole stillage is separated to obtain a solids fraction (distiller's wet grain) and a soluble fraction (thin stillage). The solids fraction is resuspended in a suitable amount of water and an effective amount of said enzyme preparation containing endoxylanase activity is added. The suspension is incubated for an appropriate time period at an appropriate temperature, and then the liquid fraction is isolated from the incubated suspension.

The US 2010-035302A method starts with the milled cereal and immediately creates a slurry, which is subjected to saccharification—no pentosan fraction is obtained. The use of a micro-organism in the method is necessary to produce the ethanol from the glucose, but is an added complication in the production of arabinoxylans. Furthermore, ethanol evaporation apparatus is costly and complex.

Both of the above described methods separate the arabinoxylans from glucose, once the starch in a wheat based slurry has been saccharified using different methods. This has, until the present invention, been considered to be the most efficient method, because the viscosity of the slurry is high, making the separation of the various phases difficult. Accordingly, the accepted methods aim to reduce the viscosity of the slurry before separation by converting the starch to glucose. Unfortunately, the viscosity is not significantly reduced, but the delivery of the arabinoxylans is delayed and requires the employment of costly and complex methods and apparatus (multi-stage filtration apparatus and/or ethanol evaporation apparatus).

It is an object of the present invention to address one or more of the problems of the prior art as discussed herein or otherwise.

Therefore, it is now desired to provide an improved method for the production of arabinoxylan-enriched preparations from a pentosan fraction derived from a wheat flour separation process, for example, high pressure disintegration (HPD) processing on wheat flour. It is also desired to provide improved processing for pentosan fractions derived from a wheat flour separation process, for example, high pressure disintegration (HPD) processing on wheat flour in order to minimise waste and produce commercially useful products.

### SUMMARY OF THE INVENTION

In a first aspect of the present invention there is provided a method for the production of arabinoxylan-enriched preparations from a pentosan fraction derived from a wheat flour comprising the following sequential steps:
mixing the pentosan fraction with water to obtain a pentosan slurry;
centrifuging the slurry to obtain liquid and solid phases; and
drying the liquid phase to provide the arabinoxylan-enriched preparation.

By arabinoxylan-enriched preparation, what is meant is a preparation or material comprising at least 30 wt % (dry) of arabinoxylans.

By ‘sequential steps’, what is meant is, the steps occur in the order provided one after the other, although the order may be interrupted with optional additional steps.

It has been found that arabinoxylans occur naturally in both soluble and insoluble form in wheat flour. The soluble arabinoxylans with a large molecular weight contribute to the high viscosity of the pentosan slurry. This has presented difficulties in past methods.

With the method of the present invention, the separation of the arabinoxylans from starch and proteins at an early stage through centrifugation significantly reduces the viscosity of the subsequently obtained starch phases, making the processing of those phases both time and cost efficient. Furthermore, by separating the pentosan slurry into liquid arabinoxylan and solid starch/protein fractions at a much earlier stage, both the liquid and solid phases can be simultaneously subjected to less complex methods to provide an enriched arabinoxylan material in addition to a liquid glucose product (for use in industrial food processes) and a protein/starch dry material (for use in animal feed).

In a second aspect of the present invention there is provided a method for the production of starch- and protein-enriched intermediates from a pentosan fraction derived from a wheat flour comprising the following sequential steps:
mixing the pentosan fraction with water to obtain a pentosan slurry;
centrifuging the slurry to obtain liquid and solid phases; and
obtaining the solid phase to provide the starch- and protein-enriched intermediate.

By starch- and protein-enriched intermediate, what is meant is a material comprising at least 40 wt % (dry) of starch and 40 wt % (dry) protein, suitable for further processing or use.

In a third aspect of the present invention there is provided a method for the production of glucose-enriched preparations from a pentosan fraction derived from a wheat flour comprising the following sequential steps:
mixing the pentosan fraction with water to obtain a pentosan slurry;
centrifuging the slurry to obtain liquid and solid phases;
liquefying the solid phase;
saccharifying the liquefied solid phase; and
fractionating the saccharified phase to obtain a glucose-enriched liquid phase.

By glucose-enriched preparation, what is meant is a preparation or material comprising at least 6 wt % (dry) of glucose.

In a fourth aspect of the present invention there is provided a method for the production of starch- and protein-enriched materials from a pentosan fraction derived from a wheat flour comprising the following sequential steps:
mixing the pentosan fraction with water to obtain a pentosan slurry;
centrifuging the slurry to obtain liquid and solid phases;
liquefying the solid phase; and
fractionating the saccharified phase to obtain a starch- and protein-enriched solid phase.

By starch- and protein-enriched material, what is meant is a preparation or material comprising at least 25 wt % (dry) starch and at least 20 wt % (dry) protein.

In a fifth aspect of the invention there is provided a method for the production of arabinoxylan-enriched preparations, starch- and protein-enriched preparations and glucose-enriched preparations from a pentosan fraction derived from a wheat flour comprising the following sequential steps:
mixing the pentosan fraction with water to obtain a pentosan slurry;
centrifuging the slurry to obtain liquid and solid phases; then
drying the liquid phase to provide the arabinoxylan-enriched preparation and liquefying the solid phase;
saccharifying the liquefied solid phase and fractionating the saccharified phase to obtain a glucose-enriched phase and a starch- and protein-enriched phase.

In the above aspects of the invention, the addition of water to the pentosan fraction reduces the viscosity of the pentosan fraction to facilitate subsequent separation steps by dissolving the soluble arabinoxylans.

Accordingly, the pentosan slurry may comprise a solid content of approximately 5 wt % to approximately 50 wt %. Preferably, the pentosan slurry comprises a solid content of approximately 10 wt % to approximately 35 wt %. Most preferably, the pentosan slurry comprises a solid content of approximately 15-30 wt %.

Preferably, the pentosan slurry is subjected to an enzyme to degrade the polysaccharides of arabinoxylan. The degradation may comprise using one or more of an endo-xylanase, or disbranching xylanase to modify the molecule of the arabinoxylan.

Preferably therefore, the pentosan slurry is subjected to an endo-xylanase. The addition of endo-xylanase has been found to significantly reduce the viscosity of arabinoxylan-containing slurry and therefore, facilitates subsequent separation steps by converting insoluble arabinoxylans into soluble arabinoxylans by breaking down the xylan backbone of the arabinoxylans, increasing the solubility of the arabinoxylans. More of the arabinoxylans can be separated from the slurry at an early stage, thereby improving the yield of arabinoxylans on the one hand and reducing the molecular weight of soluble arabinoxylan on the other hand, further reducing the viscosity of alternate starch and protein phases for ease of subsequent processing.

Preferably, the pentosan slurry is subjected to an endo-xylanase before centrifugation. The endo-xylanase may form part of an endo-xylanase preparation. The amount of endo-xylanase may be varied to achieve a required range of molecular weights of arabinoxylans.

The pentosan slurry (with or without the endo-xylanase) may be kept at approximately 10° C. to approximately 65° C.

When the pentosan slurry is subjected to the endo-xylanase, preferably, the pentosan slurry is kept at approximately 20° C. to approximately 65° C., more preferably, at approximately 35° C. to approximately 65° C. and most preferably, at approximately 40° C. or 60° C. It is to be appreciated that at approximately 40° C. a larger average molecular weight for the arabinoxylans was achieved when compared with approximately 60° C. and so, the preferred temperature depends upon the end use of the arabinoxylans-enriched preparation.

When the pentosan slurry is not subjected to the endo-xylanase, preferably, the pentosan slurry is kept at approximately 10° C. to approximately 65° C., more preferably, at approximately 20° C. to approximately 45° C. and most preferably, at approximately 20° C. to approximately 25° C.

The pentosan slurry (with or without the endo-xylanase) may be stirred for approximately 0.5 hours to approximately 6 hours. Preferably, the pentosan slurry (with or without the endo-xylanase) is stirred for approximately 1 hour to approximately 4 hours. Most preferably, the pentosan slurry (with or without the endo-xylanase) is stirred for approximately 2 hours.

The endo-xylanase may be added in an amount of approximately 0.1 ppm to approximately 300 ppm. It is to be appreciated that the amount of endo-xylanase used depends upon the desired arabinoxylans molecular weight. In one embodiment, preferably, the endo-xylanase is added in an amount of approximately 100 ppm.

The amount of endo-xylananse may depend on the amount of the pentosan slurry being processed. This will vary between laboratory testing and scaling the process up for industrial scale processing.

The centrifugation step may comprise centrifugal forces of approximately 1000 G to approximately 10,000 G. Preferably, the centrifugation step comprises centrifugal forces of approximately 3000 G to approximately 8000 G. More preferably, the centrifugation step comprises centrifugal forces of approximately 4300 G to approximately 6000 G. Most preferably, the centrifugation step comprises centrifugal forces of approximately 4500 G.

Centrifugation may be conducted for approximately 0.5 minutes to approximately 120 minutes. Preferably, centrifugation is conducted for approximately 2 minutes to approximately 100 minutes. Preferably, centrifugation is conducted for approximately 2 minutes to approximately 4-60 minutes.

The time period for centrifugation may depend on the amount of the pentosan slurry being processed. This will vary between laboratory testing and scaling the process up for industrial scale processing.

The centrifugation may be conducted at approximately 5° C. to approximately 65° C. Preferably, the centrifugation is conducted between approximately 40° C. to approximately 60° C. Most preferably, the centrifugation is conducted at approximately 40° C.

Most preferably, centrifugation is conducted at approximately 40° C. and approximately 4500 G for 2 minutes on a 500 ml sample of pentosan slurry. Again, it will be appreciated that the time may change for scaling the process up for industrial scale processing.

Preferably, the endo-xylanase is in at least the liquid phase is denatured. This is done at a stage suitable to obtain the optimum polysaccharide chain sizes for beneficial biochemical application of the arabinoxylans.

Therefore, the endo-xylanase may be denatured after the above centrifugation step. Alternatively, the endo-xylanase may be denatured after a later step.

Preferably, the endo-xylanase is denatured by subjecting the enzyme to an elevated temperature and or an altered pH for a period of time. Most preferably, the endo-xylanase is denatured by subjecting the enzyme to a temperature of between approximately 70° C. and 90° C. and/or at a pH of approximately between 4 and 11 for a time period of between approximately 10 minutes and 60 minutes.

The enzyme may therefore, be denatured during the later drying step where high temperatures are used. Preferably, however, the enzyme is denatured in a separate step following centrifugation.

Preferably, the endo-xylanase is in both the liquid phase and the solid phase is denatured.

The above centrifugation step (primary centrifugation) may produce discrete liquid, solid and intermediate phases. It has been found that the intermediate phase comprises a mixture of arabinoxylans and glucose, protein and starches. Accordingly, therefore, the primary centrifugation step may be followed by a second centrifugation step to obtain a more defined separation of liquid and solid phases.

Preferably, the second centrifugation step is conducted on the liquid and intermediate phases.

The second centrifugation step may comprise centrifugal forces of approximately 3000 G to approximately 6000 G. Preferably, the second centrifugation step comprises centrifugal forces of approximately 3500 G to approximately 5000 G. Most preferably, the second centrifugation step comprises centrifugal forces of approximately 4500 G.

Second centrifugation may be conducted for approximately 0.5 minutes to approximately 120 minutes. Preferably, second centrifugation is conducted for approximately 1 minute to approximately 40 minutes. Most preferably, second centrifugation is conducted for approximately 2-20 minutes.

The time period for second centrifugation may depend on the amount of the pentosan slurry being processed. This will vary between laboratory testing and scaling the process up for industrial scale processing.

The second centrifugation may be conducted at approximately 10° C. to approximately 65° C. Preferably, the second centrifugation is conducted at approximately 40° C. or approximately 60° C.

The second centrifugation may be conducted at a pH of approximately between pH 4 and pH 11. Preferably, the second centrifugation is conducted at approximately a pH of approximately pH 5-8.

Preferably, the resulting solid phase is added to the solid phase from the primary centrifugation for further processing. Preferably, the resulting liquid phase is used for further processing.

The denaturing of the endo-xylanase or other enzymes may be conducted after the primary or the second centrifugation step or during the second centrifugation step. Most preferably, the endo-xylanase is denatured after the centrifugation steps in the liquid phase.

The following preferences apply more specifically to the first aspect of the invention (production of the arabinoxylan-enriched preparation).

Preferably, the liquid phase is concentrated before drying. Concentration may comprise a precipitation step, i.e. the formation of a solid in a solution during a chemical reaction or natural settling or sedimentation. Preferably, the liquid phase is subjected to chemical precipitation. Preferably, the chemical precipitation comprises ethanol precipitation. Alternatively, the liquid phase may be subject to natural settling or sedimentation before drying

When chemical precipitation is used, preferably the precipitant is removed to leave behind the precipitate, of the precipitate is removed from the precipitant before drying. Preferably therefore, the ethanol is removed to leave behind the precipitate before drying of the precipitate. Preferably, the ethanol is removed by evaporation.

The precipitate or the un-precipitated liquid phase may be dried using any suitable method. Preferably, the precipitate or the un-precipitated liquid phase is dried using drum drying, spray drying or low temperature vacuum drying.

The following applies more specifically to the second aspect of the invention (production of the starch- and protein-enriched intermediate).

Preferably, the method comprises the following additional steps to provide glucose-enriched preparations:
liquefying the solid phase; and
saccharifying the liquefied solid phase;
fractionating the liquefied solid phase; and
isolating the subsequent glucose-enriched preparation.

Preferably, the method comprises the following additional steps to provide starch- and protein-enriched preparations:
liquefying the solid phase; and
saccharifying the liquefied solid phase;
fractionating the liquefied solid phase; and
isolating the subsequent starch- and protein-enriched preparation.

The following applies to the third, fourth and fifth aspects of the invention.

Preferably, the liquefying step comprises mixing the solid phase with water. Preferably, the liquefying step comprises adding water to compose between approximately 50 wt % and approximately 75 wt % of the resultant suspension. More preferably, the liquefying step comprises mixing water to compose between approximately 60 wt % and approximately 70 wt % of the resultant suspension. Most preferably, the liquefying step comprises mixing water to compose approximately 66 wt % of the resultant suspension.

Preferably, the liquefying step comprises adding α-amylase to the resultant suspension. Most preferably, the α-amylase is thermally stable. The α-amylase may be added in an amount of approximately 50 ppm to approximately 300 ppm. Preferably, the α-amylase is added in an amount of approximately 100 ppm.

Preferably, the liquefying step comprises adjusting the suspension to a pH of approximately 4.0 to approximately 8.0. More preferably, the liquefying step comprises adjusting the suspension to a pH of approximately 5.0 to approximately 6.5. Most preferably, the liquefying step comprises adjusting the suspension to a pH of approximately 5.8.

The suspension may be left for approximately 30 minutes to approximately 360 minutes. Preferably, the suspension is left for approximately 60 minutes to approximately 200 minutes. Most preferably, the suspension is left for approximately 120 minutes.

The suspension may be incubated between approximately 60° C. and approximately 99° C. Preferably, the suspension is incubated between approximately 75° C. and approximately 99° C. Most preferably, the suspension is incubated at approximately 95° C.

The suspension may be incubated in a water bath. Preferably, the suspension is subjected to some agitation during incubation. Most, preferably, therefore, the suspension is incubated in a shaker water bath.

Preferably, the saccharifying step comprises adding glucoamylase to the liquefied solution. Preferably, the glucoamylase is added to the liquefied solution in an amount of approximately 50 ppm to approximately 300 ppm. More preferably, the glucoamylase is added to the liquefied solution in an amount of approximately 80 ppm to approximately 200 ppm. Most preferably, the glucoamylase is added in an amount of approximately 120 ppm.

Preferably, the saccharifying step comprises adding proteinase to the liquefied solution. Preferably, the glucoamylase is added to the proteinase solution in an amount of approximately 50 μm to approximately 300 ppm. More preferably, the proteinase is added in an amount of approximately 80 ppm to approximately 200 ppm. Most preferably, the proteinase is added in an amount of approximately 120 ppm.

The saccharifying step may comprise adjusting the suspension to a pH of approximately 2.0 to approximately 8.0. More preferably, the saccharifying step comprises adjusting the suspension to a pH of approximately 3.0 to approximately 5.0. More preferably, the saccharifying step comprises adjusting the suspension to a pH of approximately 4.2.

Saccharification may be conducted at approximately 30° C. to approximately 80° C. Preferably, saccharification is conducted at approximately 55° C. to approximately 65° C. Most preferably, saccharification is conducted at approximately 60° C.

Saccharification may be conducted for approximately 12 hours to approximately 60 hours. Preferably, saccharification is conducted for approximately 48 hours.

It is to be appreciated that there is an inverse relationship between the temperature used and the time period in both the liquefying and saccharification steps.

Fractionation of the saccharified suspension may comprise separation of the components using filtration or centrifugation. Most preferably, the saccharified solution is centrifuged to provide solid (starch- and protein-enriched) and liquid (glucose-enriched) phases.

In the above aspects of the invention, the pentosan fraction may be obtained using high pressure disintegration (HPD) processing, although other methods may be used.

In a further aspect of the invention there is provided an arabinoxylan-enriched preparation obtained by the above methods.

In another aspect of the invention there is provided a starch- and protein-enriched intermediate obtained by the above methods.

In a further aspect of the invention there is provided a glucose-enriched preparation obtained by the above methods.

In another aspect of the invention there is provided a starch- and protein-enriched preparation obtained by the above methods.

In a further aspect of the invention there is provided a use of the arabinoxylan-enriched preparation in food, prebiotics, nutraceuticals, pharmaceuticals and food supplements.

In another aspect of the invention there is provided a use of the arabinoxylan-enriched preparation as an immune booster.

In a further aspect of the invention there is provided a use of the glucose-enriched preparation in industrial ethanol production and/or fermentation.

In another aspect of the invention there is provided a use of the starch- and protein-enriched preparation in animal feed and/or in fertilizer.

In a further aspect of the invention there is provided an apparatus for the production of arabinoxylan-enriched preparations from a pentosan fraction derived from a wheat flour comprising:
mixing equipment to obtain a pentosan slurry from the pentosan fraction;
a centrifuge in order to separate the pentosan slurry; and
a dryer to dry a liquid phase of the centrifuged pentosan slurry.

The mixing equipment may comprise a blender, which may be a household blender. Preferably, the mixing equipment comprises an industrial size mixing tank. The mixing tank may comprise a heating means.

The centrifuge may comprise an ANDREAS HETTICH ROTANTA 460R.

Preferably, the centrifuge comprises a 2-phase nozzle/de-sludging centrifuge for the efficient separation of the mixture into its two phases and the recovery of fine particles.

The feed pump may comprise a feed pump adjustable to between approximately 2 ltrs/hr and approximately 200 ltrs/hr. Flow rates may be adjusted to one of the following: 5 ltrs/hr, 10 ltrs/hr, 25 ltrs/hr, 50 ltrs/hr, 75 ltrs/hr, 200 ltrs/hr.

The dryer may comprise a spray dryer, such as a Niro ATOMIZER TYPE M-02/A, or a drum dryer, such as an Apex SSE34A, or low temperature vacuum belt dryer, such as a MJ50-5.

Preferably, the apparatus further comprises an incubator or fermentation tank in order to incubate the slurry. The apparatus may also comprise an agitator for continuous mixing of the slurry. Most preferably, the apparatus comprises a shaking water bath, such as a Grant Instrument Ltd SS40-D. The apparatus may comprise a balance tank with an agitator, heat and cool table. The balance tank may comprise a 10-200 ltr capacity.

In a yet another aspect of the invention there is provided an apparatus for the production of starch- and protein-enriched preparations and/or glucose-enriched preparation from a pentosan fraction derived from wheat flour comprising:
mixing equipment to obtain a pentosan slurry from the pentosan fraction;
a centrifuge in order to separate the pentosan slurry; and
a boiling water bath to process a liquefied solid phase of the centrifuged pentosan slurry.

The boiling water bath may be an A. GALLENKAMP & Co. Ltd bath, or comprise a balance tank with an agitator, heat and cool table. The balance tank may comprise 10-200 ltr capacity.

The apparatus may further comprise an incubator in order to incubate the liquefied solid phase. The apparatus may also comprise an agitator for continuous mixing of the slurry. Most preferably, the apparatus comprises a shaking water bath, such as a Grant Instrument Ltd SS40-D.

It is to be appreciated that the preferred and optional features described in relation to each aspect of the invention also applies to each of the other aspects of the invention.

### BRIEF DESCRIPTION OF THE DRAWINGS

For a better understanding of the invention, and to show how exemplary embodiments may be carried into effect, reference will now be made to the accompanying drawings in which:

FIG. 1 is a flow diagram showing the steps of the high pressure disintegration (HPD) process (PRIOR ART);

FIG. 2 is a flow diagram showing the steps of a method according to an embodiment of the invention;

FIG. 3 is a flow diagram showing an example method according to an embodiment of the invention; and

FIG. 4 is a plot diagram showing the differences in the viscosity of a pentosan slurry with and without added endo-xylanase;

FIG. 5 is a bar chart showing the composition of arabinoxylan-enriched material extracted with and without an endo-xylanase;

FIG. 6 is a curve diagram showing the Cumulative Molecular Weight (Mw) distribution of arabinoxylan-enriched material measured on HPLC-SEC;

FIG. 7a shows the results of the primary centrifugation stage according to the method of FIG. 2 conducted on a first 500 ml samples of a pentosan slurry at 40° C.;

FIG. 7b shows the results of the primary centrifugation stage according to the method of FIG. 2 conducted on a second 500 ml samples of a pentosan slurry at 60° C.;

FIG. 8a shows the particle size in the liquid phase of FIG. 7a; and

FIG. 8b shows the particle size in the liquid phase of FIG. 7b.

### DETAILED DESCRIPTION OF THE EMBODIMENTS

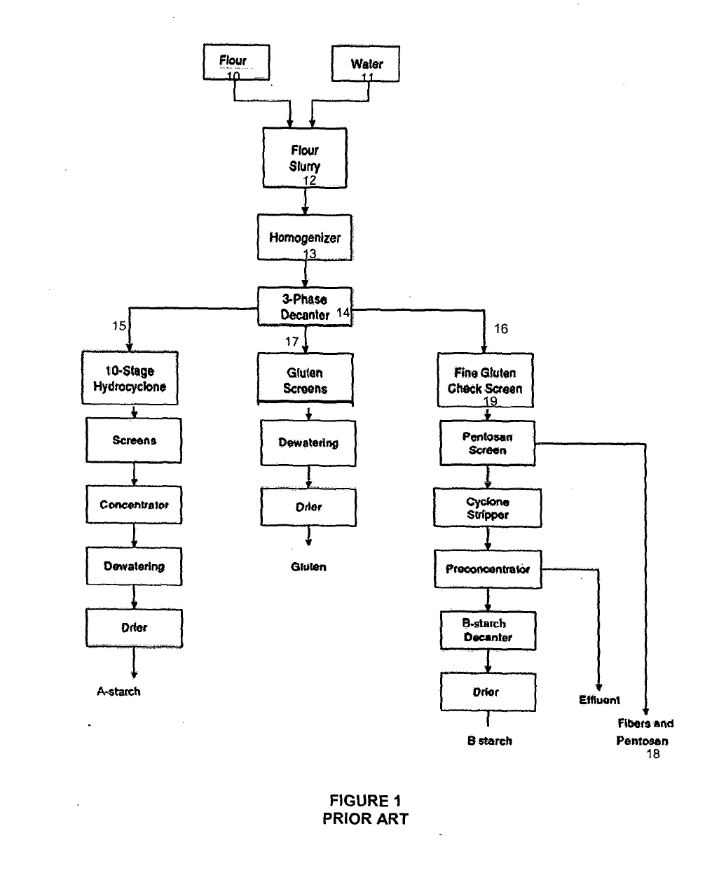

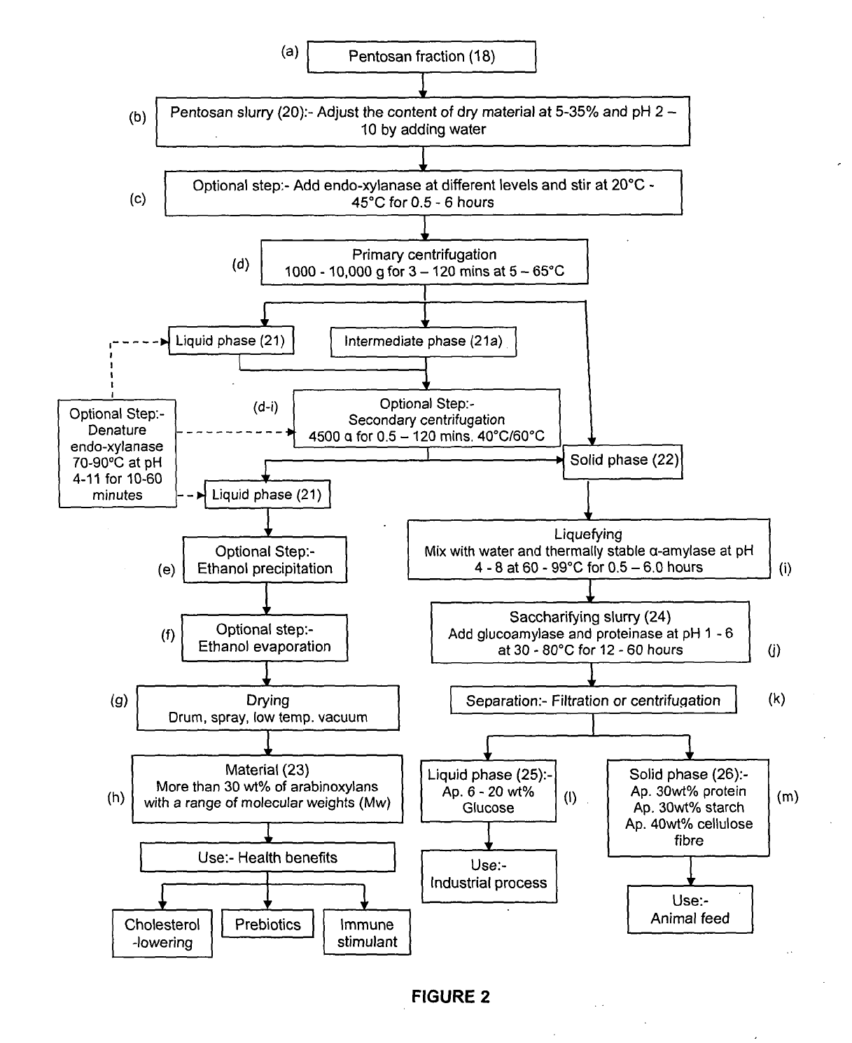

FIG. 2 shows the steps of an embodiment of the invention having obtained a pentosan fraction from high pressure disintegration (HPD) processing as shown in FIG. 1.

As shown in FIG. 1 (prior art), high pressure disintegration (HPD) processing is used for the separation of starch and gluten from wheat flour by taking advantage of their differences in solubility, density and size. Typically, a wheat flour (10) is mixed with water (11) at 35° C. in a continuous dough mixture to create a smooth slurry (12) that is then forced through a homogenising valve (13) using pressures of 100 bar. This causes starch granules to be released and gluten threads to aggregate into micrometer-sized particles. The slurry is diluted and then fed into a three-phase decanter centrifuge (14), which separates the slurry: first the A-starch stream (15) is discharged; second the B-starch and gluten are separated into two streams (16, 17) for refining; and thirdly a pentosan other solubles fraction (18) are separated from the B-starch stream (16). In the pentosan fraction (18), any remaining fine gluten particles are removed using vibrating screens (19).

As shown in FIG. 2, the pentosan fraction (18), comprising arabinoxylans, some starch, some protein and other cellulosic fibres, is utilised.

As shown in FIG. 2, according to an embodiment of the invention, the method for the production of an arabinoxylan-enriched preparation from wheat flour comprises the following steps:
separating a pentosan fraction (18) from wheat flour (10) (Step (a));
mixing the pentosan fraction (18) with water (11) to obtain a pentosan slurry (20) (Step (b));
centrifuging the slurry (20) (Step (d)); and
drying a liquid phase (21) from step (d) to provide the arabinoxylan-enriched preparation (23) (Step (g)).

It has been found that arabinoxylans occur naturally in both soluble and insoluble form in wheat flour. The soluble arabinoxylans with a large molecular weight contribute to the high viscosity of the pentosan slurry (20), which makes separation of the starch and protein complex and time-consuming.

By instead separating out of the arabinoxylans at an early stage through centrifugation the viscosity of the subsequently obtained starch phases are significantly reduced, making the processing of those phases both time and cost efficient.

Furthermore, by separating the pentosan slurry (20) into liquid arabinoxylans and solid starch/protein fractions at a much earlier stage, both the liquid (21) and solid (22) phases can be simultaneously subjected to less complex methods to provide an enriched arabinoxylan material (23) in addition to a liquid glucose product (25) (for use in industrial food processes) and a protein/starch dry material (26) (for use in animal feed).

Therefore, as also shown in FIG. 2, according to an embodiment of the present invention there is provided a method for the production of a starch- and protein-enriched intermediate (22) from wheat flour (10) comprising the following steps:
separating a pentosan fraction (18) from wheat flour (10) (Step (a));
mixing the pentosan fraction (18) with water (11) to obtain a pentosan slurry (20) (Step (b)); and
centrifuging the slurry (20) (Step (d)) to obtain the solid phase (22) to provide the starch- and protein-enriched intermediate.

Further, in FIG. 2, there is provided a method for the production of a glucose-enriched preparation (25) from wheat flour (10) comprising the following steps:
extracting a pentosan fraction (18) from wheat flour (10) (Step (a));
mixing the pentosan fraction (18) with water to obtain a pentosan slurry (20) (Step (b));
centrifuging the slurry (20) (Step (d));
obtaining the solid phase (22);
liquefying the solid phase (22) (Step (i));
saccharifying the resultant slurry (24) (Step (j)); and
obtaining the glucose-enriched preparation by fractionation of the resultant slurry (24) (Step (k)).

As shown in FIG. 2, there is also provided a method for the production of a starch- and protein-enriched preparation (25) from wheat flour (10) comprising the following steps:
separating a pentosan fraction (18) from wheat flour (10) (Step (a));
mixing the pentosan fraction (18) with water to obtain a pentosan slurry (20) (Step (b));
centrifuging the slurry (20) (Step (d));
obtaining the solid phase (22);
liquefying the solid phase (22) (Step (i));
saccharifying the resultant slurry (24); and
obtaining the starch- and protein-enriched preparation by fractionation of the resultant slurry (24) (Step (k)).

As shown in FIG. 2, the pentosan slurry (20) is adjusted to a dry material content of between 5 wt % and 35 wt % and a pH of between 2.0 and 10.0. The addition of water reduces the viscosity of the pentosan fraction (18) to facilitate centrifugation steps by dissolving the soluble arabinoxylans. Centrifugation technology is a much more time and cost efficient method for the separation of slurries when compared with multi-stage filtration and so is favoured.

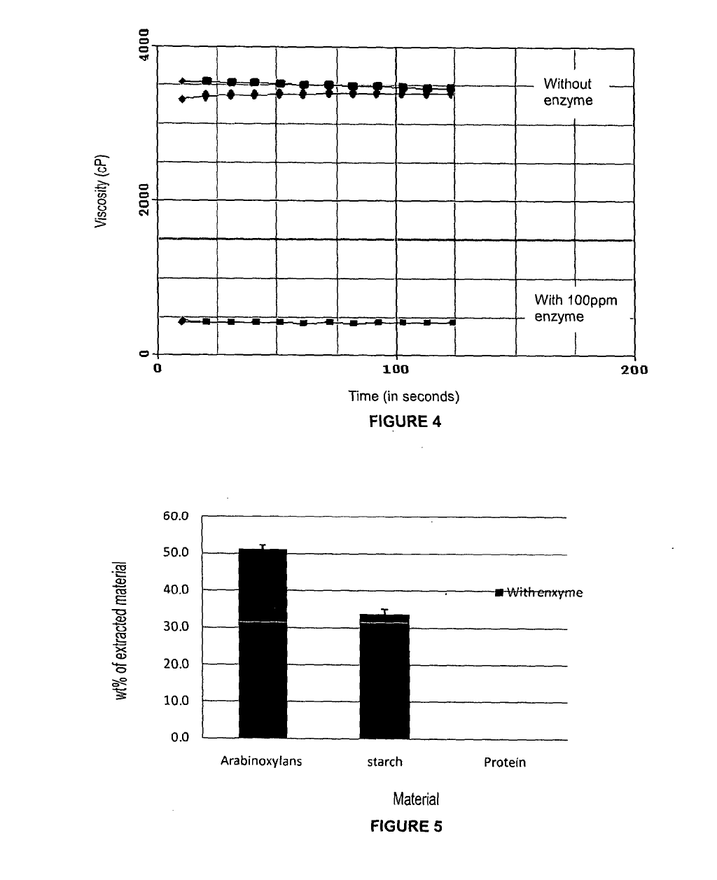

As can be seen from FIG. 2, the pentosan slurry (20) can be subjected to the optional step of adding an endo-xylanase in a further step (c) before centrifugation. Once the endo-xylanase is added, the preparation is stirred at approximately 20° C.-65° C. for a period of approximately 0.5-6.0 hours. The addition of endo-xylanase has been found to significantly reduce the viscosity of the fraction to facilitate separation steps by converting insoluble arabinoxylans into soluble arabinoxylans and high molecular weight (e.g. an average molecular weight of 2×10⁵ Da) soluble arabinoxylans into low molecular weight arabinoxylans (e.g. 1000-10,000 Da). In addition to further facilitating the separation of the slurry (20) into a liquid phase (21) and a solid phase (22) using centrifugation technology, it also improves the yield of arabinoxylans, by making sure that more of the arabinoxylans are present in the liquid (soluble) phase. This can be seen from FIG. 4, when the endo-xylanase is added, the viscosity of the slurry (20) is significantly reduced.

The amount of endo-xylanase can be varied to achieve a required range of molecular weights of arabinoxylans. Best results have been obtained when the endo-xylanase is added in an amount of approximately 0.1 ppm to approximately 300 ppm, but the eventual use of the arabinoxylan-enriched preparation dictates what is considered to be the best result. For example, arabinoxylans with a molecular weight of 1000-10,000 Da (DP=5-50 repeating units) were extracted using 150 ppm endo-xylanase, which are optimum arabinoxylans for use as prebiotics for human beings. In contrast, arabinoxylans with an average molecular weight of 2×10⁵ Da (DP=approximately 1000 repeating units) were extracted without the addition of endoxylanase. The arabinoxylans with various molecular weights have been reported to have the different bioactivities.

The primary centrifugation step comprises subjecting the slurry (20) to centrifugal forces of approximately 1000 G to approximately 10,000 G, which allows the efficient separation of the majority of the arabinoxylans from the majority of the starch and protein and other cellulosic fibres. The centrifugation in step (c) is conducted for approximately 3 minutes to approximately 120 minutes and at approximately 5° C. to approximately 65° C.

The primary centrifugation step in some instances produces liquid (21), solid (22) and intermediate (21a) phases. Depending upon the amount of intermediate phase (21a), the liquid (21) and intermediate (21a) phases can be subjected to an optional second centrifugation step (d-i) conducted for approximately 1 minute to approximately 120 minutes and at approximately 40° C. or approximately 60° C. at approximately 4500 G. The resultant solid is added to the initial solid phase (22).

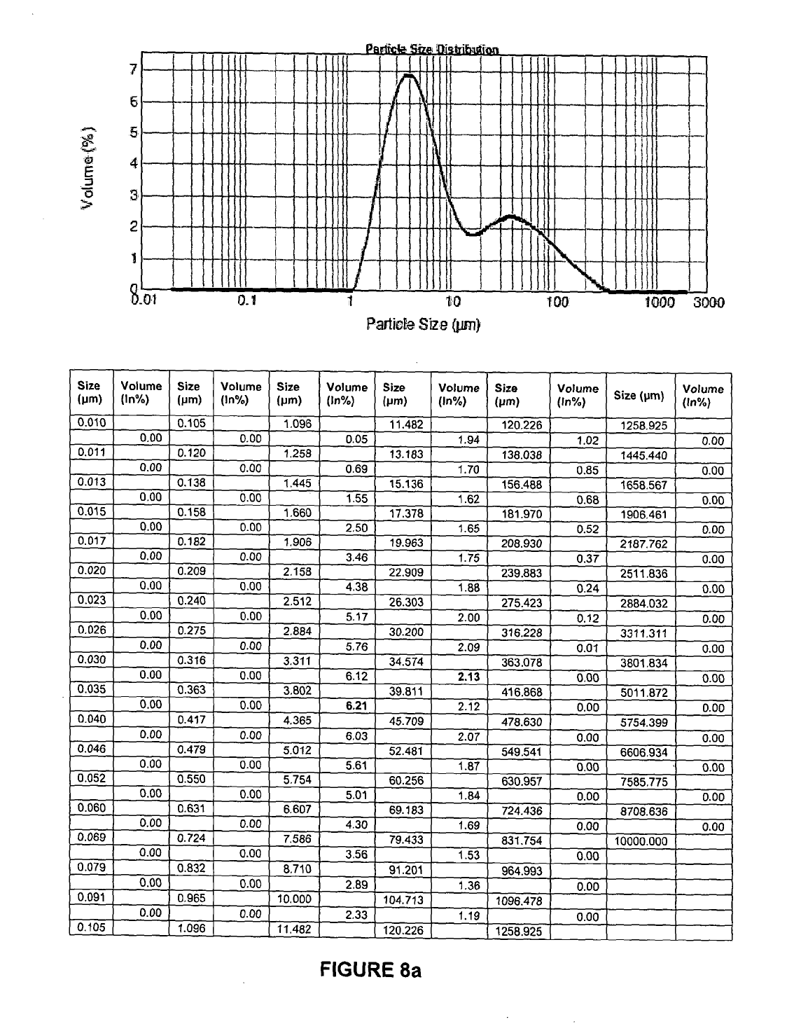

FIGS. 7a and 7b show examples of the likely liquid (21), solid (22) and intermediate (21a) phases obtained from the primary centrifugation stage (4500 G) conducted on a first 500 ml samples of a pentosan slurry at 40° C. and 60° C., respectively.

The first sample (FIG. 7a) was subjected to primary centrifugation at 40° C. for 0.5, 1 and 2 minutes respectively, The second sample (FIG. 7b) was subjected to primary centrifugation at 60° C. for 0.5, 1 and 2 minutes respectively.

The liquid phase (21) was found to comprise a soluble arabinoxylan phase, the intermediate phase (21a) comprised a mixture of arabinoxylans, proteins, starches and glucose and the solid phase (22) comprised proteins, starches and glucose.

Both sets of results showed an increase in the % amount of liquid phase (21) and a decreasing intermediate phase (21a) as time increased. In both cases, the % amount of solid phase (22) appeared to be relatively unaffected by time.

Between the two sets of results, an increase in temperature from 40° C. to 60° C. on the whole decreased the % yield of liquid phase (21), increased the % yield of unresolved intermediate phase (21a) and increased the % yield of solid phase (22).

The optimum liquid phase (21) yield was provided at 40° C. when centrifuged for 2 minutes (51%), In this result, the intermediate phase (21a) made up 35% with only 14% of solid phase.

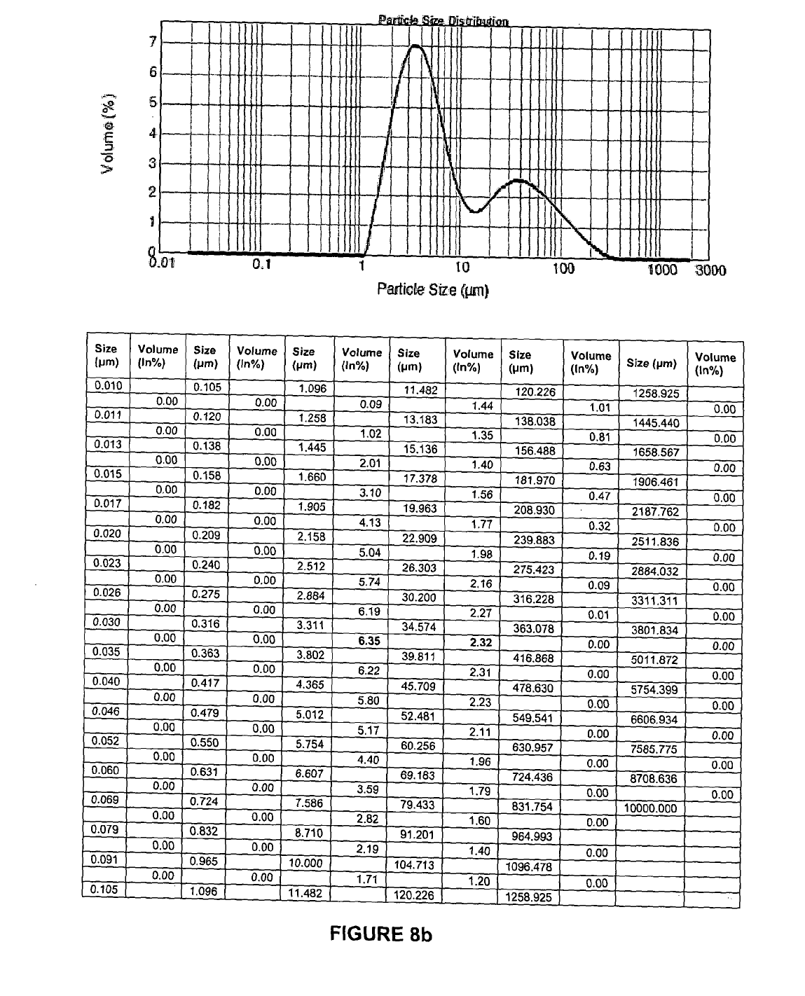

FIGS. 8a and 8b show the particle size of the liquid phase (21). It can be seen that in both samples, the particle size distribution is almost identical.

In the first sample (FIG. 8a), the particle sizes span between 1.096 μm and 316.228 μm, with a large peak at 3.802 μm and a second smaller peak at 34.574 μm. In the second sample (FIG. 8b), the particle sizes also span between 1.096 μm and 316.228 μm, with a large peak at 3.802 μm and a second smaller peak at 34.574 μm. The only relatively insignificant differences relate to the slightly higher quantities of the peaks measured in the second sample and the trough between the two peaks showing a greater drop in particles sizes between 8.710 μm and 26.303 μm in the second sample (60° C.).

The endo-xylanase in the liquid phase (21) and/or intermediate phase (21a) is denatured to avoid degradation of the arabinoxylan chains beyond an optimal size distribution. The denaturing step can be conducted by high temperatures during a later drying step. However, it is preferable to do this either soon after the primary centrifugation step (d), or during or after the second centrifugation step (d-i). These three instances are shown in FIG. 2 by dotted arrows.

Denaturing of the endoxylanase is performed by subjecting the relevant phases to temperatures of between approximately 70-90° C. at a pH 4-11 for 10-60 minutes. These conditions serve to deactivate the enzyme without being detrimental to the arabinoxylans or any other proteins which may be later separated.

As can be seen from FIG. 2, the liquid phase (21) can be subjected to the further optional step of ethanol precipitation (step (e)). This allows the arabinoxylans to settle to further increase the purity of the arabinoxylans in the material (23) by addition of ethanol up to 65 wt %

Following precipitation, the ethanol is evaporated (step (f)) to leave behind a precipitate for drying (step (g)) at approximately 45° C. in the oven.

The liquid phase (21) is then dried by drum drying techniques or spray drying techniques and the resultant material (23) comprises more than 30 wt % of arabinoxylans along with some starch and proteins and cellulosic material.

When both the optional steps (c), (e) and (f) are utilised, the yield of the arabinoxylans can be significantly increased to up to 90 wt % of the material (23) by making sure that more arabinoxylans are soluble and so migrate into the liquid phase (21) and by concentrating the liquid phase and removing a higher proportion of the remaining starches and proteins.

The arabinoxylan-enriched material (23) is suitable for a range of uses, depending upon the range of molecular weights and the yield, for example, use in prebiotics, immune stimulants, cholesterol-lowering products, as a bakery ingredient, nutraceuticals, pharmaceuticals and supplements.

The solid phase (22) from step (d) can be subject to further processing (steps (i) to (m)) simultaneously with the further processing of the liquid phase (21) if required.

The solid phase (22) is liquefied by mixing the solid phase (22) with water to create a suspension with a solid content of approximately between 25 wt % and 50 wt %. A thermally stable α-amylase is added to the suspension in an amount of approximately 50 ppm to approximately 300 ppm and the suspension is adjusted to a pH of approximately 4.0 to approximately 8.0 and a temperature of approximately 60° C. to approximately 99° C. in a shaker water bath and the suspension is left for approximately 30 minutes to approximately 360 minutes.

The suspension is subjected to a saccharifying step (step (j)) comprising adding a glucoamylase in amounts of approximately 50 ppm to approximately 300 ppm and a proteinase in an amount of approximately 50 ppm to approximately 300 ppm to the suspension. The suspension is adjusted to a pH of approximately 2.0 to approximately 8.0 and a temperature of approximately 30° C. to approximately 80° C. and is left for approximately 12 hours to approximately 60 hours in a shaker water bath.

Following the saccharifying step (step (j)), a proportion of the starch has been converted to glucose and this is separated using either filtration or centrifugation (step (k)). The liquid phase (25) of centrifugation comprises approximately 6 wt % to 20 wt % of glucose, with the solid phase (26) comprising approximately 30 wt % of protein, approximately 30 wt % of starch and approximately 40 wt % of other cellulosic fibres.

The glucose-enriched material (25) is suitable for a range of uses, but is particularly useful for industrial processes, such as ethanol production.

The solid phase material (26) meets the requirements for use in animal feed and can be easily pelletized for such use.

The whole process is straightforward and provides arabinoxylan, starch/protein and glucose preparations with acceptable yields for use in a number of products without requiring further processing. Therefore, the process produces minimum waste.

A specific embodiment of the method of the invention shall now be described.

### Example 1

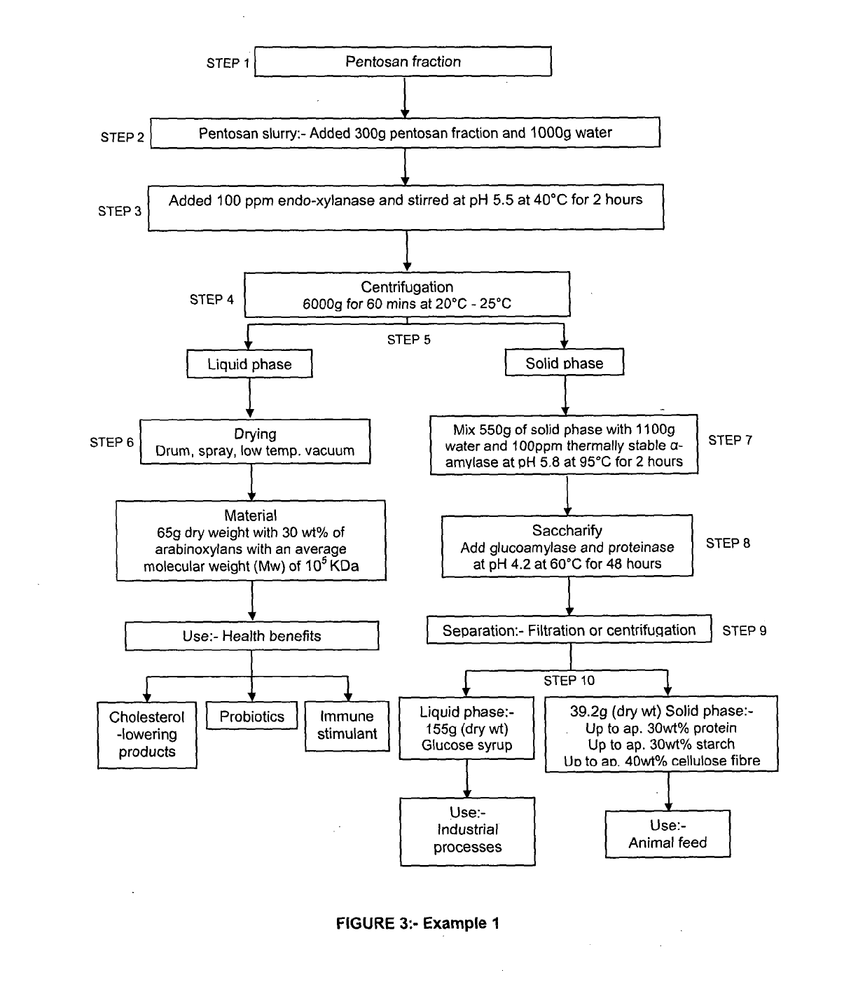

This example is shown in FIG. 3.

The following equipment was used in Example 1:

| Equipment | Model | Manufactuers |
|---|---|---|
| LABORATORY CENTRIFUGE | ROTANTA 460R | ANDREAS HETTICH GmbH & Co. |
| | | KG |
| SHAKING WATERBATH | SS40-D | GRANT INSTRUMENT Ltd. |
| BOILING WATERBATH | | A. GALLENKAMP & Co. Ltd. |
| DIGITAL BENCH-MODEL | M 220 | |
| HOUSEHOLD BLENDER | | KENWOOD |
| 10 SERIES SPECTROPHOTOMETER | 335907P-04 | THERMO ELECTRON |
| | | CORPORATION |
| ELECTRONIC ANALYTICAL BALANCE (4 DECIMALS) | AE200 | METTLER-TOLEDO LTD. |
| HF SERIES MULTI-FUNCTION PRECISION BALANCE | HF 3000 | A&D INSTRUMENTS, A&DCO. |
| (2 Decimals) | | Ltd. |
| LABORATORY DRYING OVEN | T20 | KENDRO LABORATORY |
| | | PRODUCTS GmbH |
| MOBILE MINOR SPRAY DRYER | ATOMIZER TYPE | NIRO ATOMIZER Ltd. |
| | M-02/A | |
| APEX SINGLE DRUM DRYER | SSE34A | APEX CONSTRUCTION Ltd. |

Step 1:—The pentosan fraction (18) was obtained from a high pressure disintegration (HPD) process as shown in FIG. 1 (described above).

Step 2:—300 g of the pentosan fraction (18) was mixed with 1000 g of water to create pentosan slurry comprising approximately 23 wt % of pentosan fraction and approximately 77 wt % of water.

Step 3:—endo-xylanase was added to the slurry in an amount of 100 ppm and pH was adjusted to approximately 5.5. The slurry was stirred for approximately 2 hours at a temperature of approximately 40° C. in order to convert insoluble arabinoxylans into soluble arabinoxylans. After this stage, it was noted that the viscosity had decreased from above 3000 cP to below 500 cP (FIG. 4).

Step 4:—the slurry was cooled and then centrifuged with forces of approximately 6000 G for approximately 60 minutes and at room temperature (20-25° C.).

Step 5:—700 ml of liquid phase was decanted from the 550 g of solid phase (192 g in dry weight).

Step 6 and steps 7 to 11 were performed consecutively, but it is to be understood that they could be performed in two simultaneous processing pathways, especially when considering application on an industrial scale.

Step 6:—The liquid phase was dried on a lab scale drum drier to provide 65 g (dry weight) of arabinoxylan-enriched material.

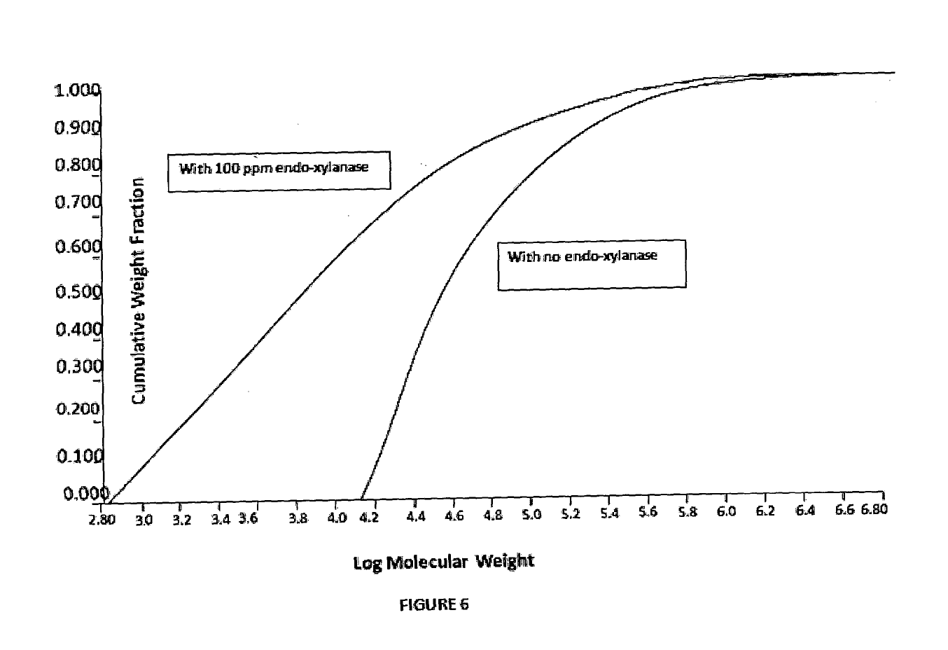

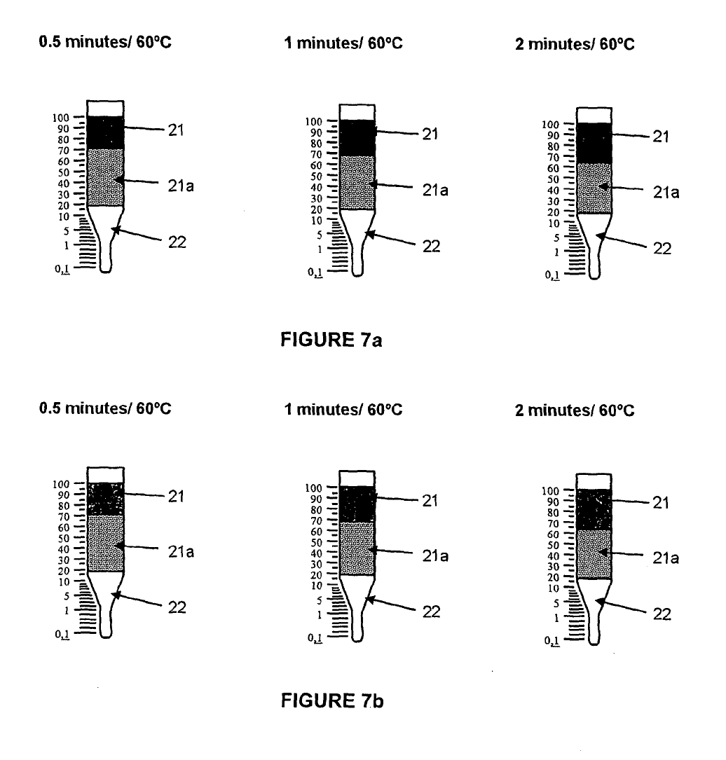

Analysis of the composition revealed that the arabinoxylan content of the material was 51.2 wt % (compared with 15 wt % of the original pentosan fraction in step 1) (FIG. 5). Starch made up 33.2 wt % of the material. Further analysis revealed that the molecular weight distribution of arabinoxylans measured on HPLC-SEC (FIG. 6) was 50% arabinoxylans with a molecular weight bigger than 10⁵ Da and 5% of arabinoxylans with a molecular weight bigger than 10⁶ Da.

Step 7:—The 550 g solid phase was mixed with 1100 g of water to create a slurry comprising approximately 33.3 wt % of solid material in approximately 66.6 wt % of water. A thermally stable α-amylase was added to the slurry in an amount of approximately 100 ppm and the pH was adjusted to approximately 5.8. The slurry was left at approximately 95° C. for approximately 2 hours.

Step 8:—Glucoamylase and proteinase were added to the slurry in an amount of approximately 100 ppm each and the pH was re-adjusted to approximately 4.2. The slurry was left at approximately 60° C. for approximately 48 hours.

Step 9:—the slurry was subjected to centrifugation at approximately 6000 g for approximately 2-60 minutes and at room temperature (20-25° C.).

Step 10:—The liquid phase was decanted from the solid phase.

The further liquid and solid phases were analysed and were found to provide the following components:—

| Component | Dry weight (in g) |
|---|---|
| Glucose (liquid phase) | 155.8 |
| Residue (solid phase) | 39.3 |

The glucose was found to be suitable for direct use in any fermentation process.

The 39.3 g of residue product was further analysed and found to contain 23.3 wt % protein and 23.8 wt % of starch with other cellulosic materials and so, was found to meet the requirements for animal feed or fertilizer.

From 300 g of pentosan fraction, the following materials were extracted:—

| Component | Dry weight (in g) |
|---|---|
| Arabinoxylan-enriched material | 65.0 |
| Glucose (further liquid phase) | 155.8 |
| Residue (solid phase) | 39.3 |

Previously utilised methods separate the arabinoxylans from glucose, once the starch in the pentosan slurry has been saccharified. This has, until the present invention, been considered to be the most efficient method due to a recognition that the pentosan slurry is difficult to process. Accordingly, the accepted methods aim to convert the starch to glucose in order to reduce the viscosity of the slurry before separation. The viscosity is somewhat reduced, but the delivery of the arabinoxylans is delayed and requires the employment of costly and complex methods and apparatus (multi-stage filtration apparatus and/or ethanol evaporation apparatus) to separate the arabinoxylans from glucose.

In contrast with previous methods, in the method of the present invention, the separation out of the arabinoxylans at an early stage through centrifugation significantly reduces the viscosity of the subsequently obtained starch phases, making the processing of those phases both time and cost efficient. Furthermore, by separating the pentosan slurry into liquid arabinoxylan and solid starch/protein fractions at a much earlier stage, both the liquid and solid phases can be simultaneously subjected to less complex methods to provide an enriched arabinoxylan material in addition to a liquid glucose product (for use in industrial food processes) and a protein/starch dry material (for use in animal feed).

Therefore, the method of the present invention avoids the use multi-stage membrane filtration of glucose and arabinoxylans, which is complex, costly and time-consuming and delays the delivery of the arabinoxylans. Nor does the method of the present invention require the use of a micro-organism, which is an added and unnecessary complication in the production of arabinoxylans. As ethanol evaporation apparatus can be costly and complex, the necessity for this is removed to provide high yield products, but can be included if the highest yields are desired.

Therefore, the present invention provides an improved method for the production of arabinoxylan-enriched preparations from a pentosan fraction derived from a wheat flour separation process, for example, high pressure disintegration (HPD) processing on wheat flour. The present invention also provides an improved method for processing pentosan fractions derived from a wheat flour separation process, in order to minimise waste and produce commercially useful products.

It will be appreciated that the above example was conducted in a laboratory using laboratory apparatus and equipment. However, it is to be appreciated that the method could be scaled-up for use on an industrial scale.

Although a few preferred embodiments have been shown and described, it will be appreciated by those skilled in the art that various changes and modifications might be made without departing from the scope of the invention.

1. A method for the production of arabinoxylan-enriched preparations from a pentosan fraction derived from a wheat flour comprising the following sequential steps:
mixing the pentosan fraction with water to obtain a pentosan slurry;
centrifuging the slurry to obtain liquid and solid phases; and
drying the liquid phase to provide the arabinoxylan-enriched preparation.

2.-5. (canceled)

6. A method according to claim 1, wherein the pentosan slurry comprises a solid content of approximately 5 wt % to approximately 50 wt %.

7. A method according to claim 1, wherein the pentosan slurry is subjected to enzymes to at least one of reduce the molecular weight and modify the structure of the arabinoxylans.

8. A method according to claim 6, wherein the pentosan slurry is subjected to an endo-xylanase.

9. A method according to claim 8, wherein the pentosan slurry is kept at approximately 10° C. to approximately 65° C. and the pentosan slurry is stirred for approximately 0.5 hours to approximately 6 hours.

10. (canceled)

11. A method according to claims 7 to 9, wherein the endo-xylanase or other enzymes are added in an amount of approximately 0.1 ppm to approximately 300 ppm.

12. A method according to claim 1, wherein the centrifugation step may comprise centrifugal forces of approximately 1000 G to approximately 10,000 G at approximately 5° C. to approximately 65° C.

13. (canceled)

14. A method according to claims 7 to 9, wherein the endo-xylanase or other enzymes are, in at least the resultant liquid phase, denatured by subjecting the enzyme to an elevated temperature and/or an altered pH for a period of time.

15. (canceled)

16. A method according to claim 1, wherein when the centrifugation step produces liquid, solid and an intermediate phase, the centrifugation step is followed by a second centrifugation step on the combination of liquid and intermediate phases.

17. A method according to claim 16, wherein the second centrifugation step comprises centrifugal forces of approximately 3000 G to approximately 6000 G at approximately 35° C. to approximately 65° C.

18. (canceled)

19. A method according to claims 16 or 17, wherein the resulting solid phase is added to the solid phase from the centrifugation initially performed for further processing.

20. A method according to claim 1, wherein the liquid phase is concentrated before drying.

21. A method according to claim 20, wherein the liquid phase is subjected to chemical precipitation.

22. A method according to claim 1, wherein the method comprises the following additional steps to provide a glucose-enriched, starch-enriched and protein-enriched preparations:
liquefying the solid phase; and
saccharifying the liquefied solid phase;
fractionating the liquefied solid phase; and
isolating the subsequent glucose-enriched, starch-enriched and protein-enriched preparations.

23. (canceled)

24. A method according to claim 22, wherein the liquefying step comprises mixing the solid phase with water to compose between approximately 50 wt % and approximately 75 wt % of the resultant suspension.

25. A method according to claim 24, wherein the liquefying step comprises adding α-amylase to the resultant suspension.

26.-27. (canceled)

28. Use of an arabinoxylan-enriched preparation in one or more of: food, immune stimulants, prebiotics, nutraceuticals, pharmaceuticals and food supplements.

29.-30. (canceled)
